(19) United States
(12) Patent Application Publication
(10) Pub. No.: US 2005/0277833 A1
(43) Pub. Date: Dec. 15, 2005

(54) System, method, and computer program product for handling, mixing, dispensing, and injecting radiopharmaceutical agents

(75) Inventor: Robert C. Williams JR., Fort Salonga, NY (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: E-Z-EM, Inc.

(21) Appl. No.: 11/140,617

(22) Filed: May 27, 2005

Related U.S. Application Data
(60) Provisional application No. 60/574,875, filed on May 27, 2004.

(51) Int. Cl. A61B 6/00; A61N 5/00

(52) U.S. Cl. 600/431; 600/4

(57) ABSTRACT

The present invention is directed to a system, method, and computer program product for handling, mixing, dispensing and/or injecting a mixture into an individual during a medical procedure. The present invention provides one or more mixing devices, containers, and dispensing devices to facilitate the handling, mixing, dispensing, and/or injecting of a mixture containing, for example, pharmaceutical agents and/or radiopharmaceutical agents. The present invention also provides a mixing device capable of diluting a radiopharmaceutical agent with, for instance, a diluent, for altering a radiation dose emitted by the radiopharmaceutical agent.

### CROSS-REFERENCE TO RELATED APPLICATION

This application claims the benefit of U.S. Provisional Application No. 60/574,875, filed May 27, 2004, which is incorporated herein in its entirety.

### FIELD OF THE INVENTION

The present invention relates generally to handling, mixing, and/or dispensing systems and methods. More particularly the present invention relates to a system, method, and/or computer program product for the handling, mixing, dispensing, and/or injection of a mixture containing, for example, radiopharmaceutical agents, for use in various types of diagnostic imaging and/or therapeutic procedures.

### BACKGROUND OF THE INVENTION

Emissions from radioactive sources are often used in the medical field to provide imagery of internal body structures including, but not limited to, bone, vascular, organ systems, and other tissue. In addition, such emissions may also be used as therapeutic agents to inhibit the growth of targeted cells or tissue, such as, for instance, cancer cells. In addition, some pharmaceutical agents and/or radiopharmaceutical agents having hazardous physical and/or chemical effects when exposed to individuals (including, but not limited to, clinicians, imaging technicians, and pharmacists) are also often used in the medical field in therapeutic, diagnostic, and/or other medical procedures.

According to conventional radiographic diagnostic imaging techniques, such as X-ray procedures, X-rays pass through a target object and expose an underlying photographic film. The developed film then provides an image of the radiodensity pattern of the object. Less radiodense areas produce a greater blackening of the film; more radiodense, bony tissues produce a lighter image. Effective contrast agents for X-ray may be either less radiodense than body tissues or more radiodense. The less radiodense agents include, for example, air and other gases; an example of a more radiodense contrast material is a barium sulfate suspension.

Computed tomography (CT) is superior to conventional radiography in its ability to image, with extremely high resolution, a succession of thin sections of an object at specific points, lines or planes along the X, Y, or Z axis of the target object. However, because this procedure is also based on the detection of differences in radiodensity, requirements for contrast agents in CT are essentially identical with those for conventional radiography.

Magnetic resonance imaging (MR) systems for body imaging operate on a different physical principle. Generally, MR relies on the atomic properties (nuclear resonance) of protons in tissues when they are scanned with radio frequency radiation. The protons in the tissue, which resonate at slightly different frequencies, produce a signal that a computer uses to tell one tissue from another. MR can provide detailed three-dimensional soft tissue images.

Other imaging methods, however, that are used to obtain information about function-related tissues, may use radiopharmaceutical agents and/or other pharmaceutical agents as tracers to interact with the targeted tissues. These methods include, but are not limited to, procedures such as single photon emission computerized tomography (SPECT) and positron emission tomography (PET). SPECT uses a molecule normally found in the body in which one of the atoms of the molecule is replaced by a radioactive atom contained within a radiopharmaceutical agent that is injected into the individual. The radiopharmaceutical agent, which is chosen for its ability to interact with specific tissues, is sometimes called a tracer. The tracer emits photons that can be detected as the tissue is scanned at various angles or as the photons pass through a detector array. In certain embodiments, a computer reconstructs a 3-dimensional color tracer image. PET uses radiopharmaceutical agents as tracers to produce 3-D color images with a greater sensitivity than with SPECT. PET can be used in combination with CT to create a complimentary imaging effect in an imaging technique called CT-PET.

The radioactivity levels of the radiopharmaceutical agents used as tracers in, for instance, SPECT and PET procedures, are measured by medical personnel such as radio-pharmacists, to determine the radiation dose that the individual will receive during the course of a diagnostic procedure. The radiation dose received depends on a number of factors, including the half-life of the radiopharmaceutical agent (which, in turn, determines the total time the individual is exposed to radiation from the radiopharmaceutical agent), and the initial radioactivity level of the radiopharmaceutical agent at the time it is injected into the individual.

In PET imaging, an injectable radiopharmaceutical agent such as, for instance, FDG (fluorodeoxyglucose), is fabricated in a cyclotron device. Thereafter, the FDG may be transferred in a container device that may further comprise, for instance, an inner container device and a shielding, to prevent unnecessary radiation exposure to personnel, such as the radio-pharmacist, responsible for transporting/handling the FDG from the cyclotron to the PET imaging site. Since the half-life of FDG is short, approximately 110 minutes, it is necessary to quickly transport the FDG to the PET imaging site. Depending upon the elapsed transport time and the initial radioactivity level of the FDG at the time of fabrication, it is often required that the radioactivity level needs to be re-measured at the PET imaging site. Should a specific initial FDG radioactivity level, typically expressed milliCuries/milliliter be required at the time of patient injection, a radio-pharmacist at the PET imaging site may dilute the raw FDG with a diluent such as, for instance, IV saline solution, prior to loading the injection device with a specified volume. During this process, the handling of the FDG from container device to injection device for patient injection may be entirely manual. Within this process, several products are currently marketed to aid in shielding individuals from FDG during handling and dose calibration (measuring radiation). Although shielding may reduce the radiation exposure of the radio-pharmacist in handling the shielded vial, the radio-pharmacist may still be exposed to emissions from the radiopharmaceutical agent during the manual mixing and/or dilution process required to obtain the required dose. In addition, in some medical procedures, pharmaceutical agents or other materials emanating toxic and/or otherwise harmful emissions may be suitable for dispensing into an individual for diagnostic, therapeutic, and/or other medical procedures. It may be preferable, however, to shield individuals administering such procedures (including, but not limited to clinicians, pharmacists, and technicians), from the harmful emanations of such agents and/or materials.

Thus, there exists a need for a system, method, and/or computer program product for handling, mixing, dispensing, and injecting a mixture containing a first material, such as, for instance, a radiopharmaceutical agent and a second material, such as, for instance, an intravenous saline solution, such that an operator of such a system (e.g., a radio-pharmacist, clinician, or other individual) is subjected to reduced exposure to and/or reduced handling of the first material or mixtures formed that may contain the first material. In addition, there exists a need for a system and method for automatically mixing, diluting, and/or dispensing into an injection device such that the mixture containing, for instance, a radiopharmaceutical agent, provides a selected radiation dose amount when injected into an individual.

### SUMMARY OF THE INVENTION

In at least one alternative embodiment, the present invention provides a system comprising one or more container devices for holding a first set of one or more materials and one or more dispensing devices for holding a mixture of at least a portion of the first material and a set of one or more second materials, and other substances as well. The system of the present invention may also comprise one or more mixing devices or automated injector devices for receiving the containers so as to reduce the handling of the first material contained in the containers, for example. The mixing device may be further capable of mixing at least a portion of the first material with at least a portion of the second material according to a predetermined ratio to form a mixture. The mixing device may also direct the mixture to the dispensing device for dispensing one or more mixtures into an individual. In some embodiments of the present invention, the mixing device may be integrated with an automated injector device such that the power injection device may receive the one or more container devices to mix at least a portion of the first material with at least a portion of the second material according to a predetermined ratio to form the one or more mixtures that may then be automatically injected into an individual.

According to other aspects of the present invention, the present invention may further comprise one or more second containers for holding the second material, and a computer device operably engaged with the mixing device and configured to cooperate with the mixing device to produce the mixture according to the predetermined ratio. In other embodiments, the one or more container devices and the one or more dispensing devices may each further comprise one or more shieldings for shielding operators from radiation or other caustic or hazardous emissions that may emanate from the first or second materials and the resulting mixtures, respectively.

Another embodiment of the present invention comprises a method and/or computer program product for forming a mixture. Such a method may comprise steps for receiving at least a first container suitable for holding a first set of one or more materials at at least one mixing device, wherein the mixing device is capable of receiving the first container so as to reduce the manual handling of the first material that may be contained therein. At least a portion of the first material is mixed with at least a portion of a second material according to a predetermined ratio to form the mixture using the mixing device, the mixing device being further suitable for receiving one or more dispensing devices suitable for holding the mixture. The mixture formed by the mixing device is then directed to the one or more dispensing devices, wherein the one or more dispensing devices are suitable for dispensing the mixture so as to reduce the handling of the mixture contained within the dispensing device.

In other embodiments, the method of the present invention may further comprise the steps of dispensing the mixture to an individual using the dispensing device, inputting a predetermined radiation dose amount into a computer device operably engaged with the mixing device, converting the radiation dose amount into the predetermined ratio of first material to second material in the mixture, and injecting the mixture into an individual. The present invention may comprise mixing multiple materials in various combinations.

Embodiments of the present invention may also include a system and method whereby a mixture comprising at least a portion of a first and a second material is formed and transferred to a dispensing device wherein an operator using the system is minimally exposed to the first material and/or mixture formed by the mixing device of the system. Some embodiments of the present invention also provide a system wherein a computer device, in communication with a mixing device, may form a mixture by mixing a first material comprising, for instance, a radiopharmaceutical agent, and a second material, serving as a diluent, wherein the mixture is automatically formed having a predetermined radiation dose amount that is based on a predetermined ratio of the first material to the second material in the mixture.

The present invention also includes a mixture formed by the method embodiments described above including the steps of: operably engaging one or more containers, each one or more container holding one or more materials, with a mixing device, so as to supply the one or more materials to the mixing device and reduce handling of the one or more materials contained in the one or more containers; mixing, with the mixing device, one or more materials according to a predetermined ratio to form the mixture; and directing and/or situating the mixture relative to a dispensing device. Furthermore, according to other embodiments of the present inventions a method for preparing a radiopharmaceutical agent for injection into an individual is provided, which first comprises engaging one or more containers suitable for holding one or more materials, the one or more materials comprising one or more radiopharmaceutical agents, with a mixing device, so as to supply the materials to the mixing device and reduce handling of the materials contained in the one or more containers. The embodiment for preparing a radiopharmaceutical agent for injection further comprises the steps of: mixing, with the mixing device, the one or more materials according to a predetermined ratio to form the mixture; and directing the mixture to a dispensing device, the dispensing device being adapted for dispensing the mixture into an individual.

Such embodiments provide significant advantages as described and otherwise discussed herein.

### BRIEF DESCRIPTION OF THE DRAWINGS

Reference will now be made to the accompanying drawings, which are not necessarily drawn to scale. The drawings are for illustrative purposes only, and are not intended to limit the scope of the present invention.

FIG. 1 shows a non-limiting schematic of a system for forming a mixture, including a container device, mixing device, and dispensing device, according to one embodiment of the present invention.

FIG. 2 shows a non-limiting schematic of the present system for forming a mixture, according to one embodiment of the present invention showing the dispensing device configured as an injection device compatible with, for instance, a power injector.

FIG. 3 shows a non-limiting schematic of the present system for forming a mixture including a mixing device substantially integrated with an automated injector device.

FIG. 4 shows a non-limiting flow diagram according to the present method and computer program product for mixing a first material with a second material to form a mixture according to one embodiment of the present invention.

FIG. 5 shows a non-limiting flow diagram according to the present method and computer program product for mixing a first material with a second material to form a mixture according to one embodiment of the present invention, including the step of dispensing the mixture to an individual.

FIG. 6 shows a non-limiting flow diagram according to a method and computer program product for mixing multiple substances to form a mixture according to one embodiment of the present invention, including the steps of receiving dosage information and determining a predetermined ratio of each substance relative to each other in the mixture based on the dosage information.

FIG. 7 shows a non-limiting flow diagram according to a method and computer program product for mixing a first material with a second material to form a mixture according to one embodiment of the present invention, including the steps of determining the radioactivity level of the first material and the mixture.

FIG. 8 shows a non-limiting flow diagram according to a method and computer program product according to one embodiment of the present invention, wherein the method and computer program product are suitable for mixing a radiopharmaceutical device with a diluent such as, for example, IV saline solution, according to a predetermined ratio.

### DETAILED DESCRIPTION OF THE INVENTION

The present invention will be described with reference to the accompanying drawings, where applicable. It is understood that the present invention may be embodied in many different forms and should not be construed as limited to the embodiments set forth herein; rather, these embodiments are provided for illustrative purposes only. Like numbers refer to like elements throughout.

While the embodiments of the system and method for handling, mixing, dispensing and/or injecting mixtures including, for instance, radiopharmaceutical agents, are described below in the context of PET imaging techniques utilizing FDG as the first material, it should be understood that the embodiments of the present invention may also be utilized to handle, mix, dispense, and/or inject a variety of substances such that the substances are diluted, mixed, and/or manipulated such that the first and second materials are mixed according to a predetermined ratio to produce a mixture characterized by, for instance, a selected radiation dose amount. Further, the mixtures formed by the various embodiments of the present invention may be utilized in a variety of different imaging and/or therapeutic procedures requiring a mixture comprising a, pharmaceutical agent, for example.

As used herein, the term “material” may include, but it not limited to, one or more components which may include, but are not limited to, pharmaceutical agents, radiopharmaceutical agents, therapeutic agents, diagnostic agents, chemical compounds, diluents, flushing media, contrast media, or other materials that may be suitable and/or necessary for use in therapeutic, diagnostic, and/or medical procedures.

As used herein, the term “mixture” includes, but is not limited to, a combination, fusion, and/or blend of one or more of the materials or components described herein. According to some embodiments of the present invention, the mixture may be formed by physically and/or chemically mixing one or more of the materials or components described herein. Thus, “mixtures” of the present invention may include, but are not limited to, physical or chemical combinations of materials or components.

According to some embodiments, “radiopharmaceutical agents” may comprise radioactive materials capable of emitting radiation that may be harmful to individuals administering such material if the material is not shielded to reduce the amount of emitted radiation. Further, in some embodiments, pharmaceutical agents may also comprise toxic, caustic, and/or otherwise hazardous compounds such that the handling of such pharmaceutical agents by individuals should be reduced whenever possible.

The term “radiopharmaceutical agent” also includes, but is not limited to, a material, mixture, and/or pharmaceutical agent emitting radiation therefrom and/or providing a quantifiable radiation dose to an individual exposed thereto. Radiopharmaceutical agents may be capable of emitting radiation for treating a medical condition (for example, such as a cancerous tumor), diagnosing a medical condition (for example, by providing a radioactive marker that is detectable by an imaging or other diagnostic device), or providing images of a patient as part of a medical imaging procedure (for example, by acting as a radioactive marker or contrast media). The radiopharmaceutical agents of the present invention may be administered to an individual via injection, ingestion, or other suitable means. The term “radiation dose” includes, but is not limited to, an amount of radiation absorbed by an individual during a therapeutic, diagnostic, and/or medical procedure, wherein the radiation does may be measured using units of measure that may include, but are not limited to rem, Roentgen, curies, and/or other suitable units of measure for radiation dosage. According to embodiments of the present invention, a predetermined radiation dose may be calculated by a computer device with input from an individual in order to prepare a radiopharmaceutical agent capable of providing the predetermined radiation dose.

As used herein, the term “diluent” includes, but is not limited to, a material that may comprise liquid, solid, or gaseous materials suitable for dilution of one or more pharmaceutical agents, mixtures, or materials. Diluents may comprise various materials including, but not limited to, water, saline solutions, flushing media, intravenous solutions, or other materials suitable for diluting, flushing, or diminishing the effects of a pharmaceutical agent. In some embodiments, the diluent may be physically and/or chemically mixed with a radiopharmaceutical agent to prepare a mixture capable of providing a predetermined radiation dose when administered to an individual.

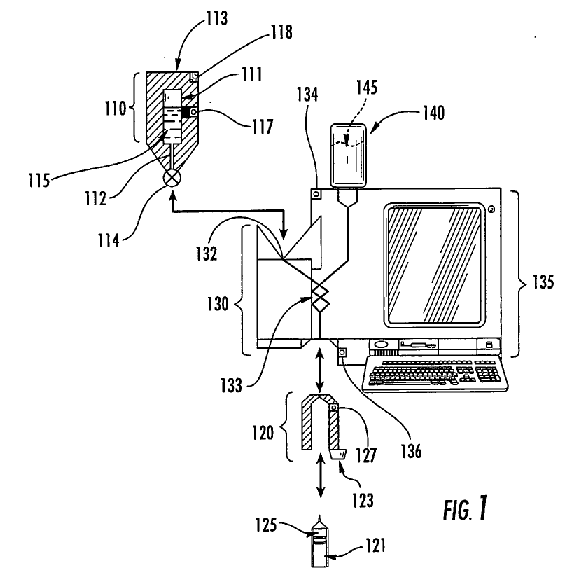

FIG. 1 shows a system for forming a mixture according to one embodiment of the present invention. The system may comprise a container 110 configured for holding a first material 115, wherein the first material, may comprise, for instance, a radiopharmaceutical agent such as FDG, which may, in turn, produce a radiation emission. Such a first material may be produced by, for instance, a cyclotron or irradiating device, such that the container device 110 may be suitable for holding and transporting the first material 115 from a cyclotron or irradiating device to the system of the present invention.

The container device 110 may also comprise shielding 113 including, but not limited to, a lead, tungsten, polycarbonate layer, or any other layer having a thickness suitable for reducing a radiation dose received by an individual operating the present invention, wherein the radiation dose may be produced by the first material 115. One skilled in the art will appreciate that the shielding 113 may be accomplished in various manners and the shielding 113 may be altered in material, thickness, number of shielding components, and other parameters in order to provide an individual with appropriate protection from a variety of radiation types, including, but not limited to, x-rays, beta radiation, gamma radiation, and other forms of radiation, depending on the characteristics of the first material 115 contained within the container device 110. One skilled in the art will appreciate that the shielding 113 may, in some cases, be incapable of completely eliminating the absorption of radiation by an individual, however, according to the various embodiments of the present invention, the shielding 113 is suitable for reducing, and in some cases minimizing and/or eliminating the radiation dose received by an individual from a material placed within the container device 110. The shielding 113 may also be suitable for protecting an individual from other toxic and/or hazardous effects of the materials, in addition to radiation. For example, the shielding 113 may provide means for reducing an individual's exposure to toxic and/or hazardous effects that may include, but are not limited to, toxic fumes, caustic materials, and/or otherwise harmful emissions that may emanate from materials contained within the container device 110.

According to one embodiment, the container 110 may further comprise an inner container 111 for holding the first material 115. In this embodiment, the inner container 111 may be placed within the shielding 113 of the container 110 so as to provide shielding from radiation emissions that may be produced by the first material 115 which may further comprise one or more radiopharmaceutical agents or other pharmaceutical agents. The inner container 111 may further comprise a material, including but not limited to, for example, a vial composed of polymer, glass, metal, or other material suitable for containing the first material 115. Furthermore, the inner container 111 may be, in some embodiments, reusable for multiple mixing iterations in the present invention. According to other embodiments, the inner container 111 may be discarded and replaced after the first material 115 is removed from the container device 110 as described below. Furthermore, according to some embodiments, the container device 110 may be provided without an inner container 111, such that the first material 115 is held directly within the container 110. Further, in alternative embodiments, the container 110 may be provided with multiple inner containers 111 that may comprise additional material layers suitable for shielding an individual from the first material 115 or emissions emanating therefrom. According to embodiments of the present invention, the container device 110 (and inner container 111, where applicable) may be suitable for minimizing handling of the first material 115 by a clinician or other operator of the present invention. Such embodiments may be especially suitable for minimizing individuals' handling of toxic, caustic, and/or otherwise harmful materials that may be components of the first material 115.

As shown in FIG. 1, the container 110 may also further comprise an aperture 112 and valve 114 configured to provide a conduit such that the first material 115 contained therein may be removed or otherwise directed from the container device 110 when the container device 110 is received by the mixing device 130 as described below. The valve 114 may be located on the exterior of the shielding 113 as shown in FIG. 1. In other embodiments, the valve 114 may be located on the interior of the shielding 113 in communication with an opening defined in the surface of the inner container device 111. The valve 114 may further comprise, for instance, a solenoid valve or other electromechanical mechanism, such that the valve 114 may be actuated remotely in a manner such that an operator's exposure to the first material 115, (via handling or via a radiation dose from the first material 115) is reduced. Alternatively, the valve 114 could be a manually actuated mechanical device such as a ball valve, stop cock or other similar device.

The container 110 and/or mixing device 130, according to other embodiments of the present invention, may be operably engaged with a measuring device 117 (such as, for example, a first radiation measurement device, or dosimetry device 117) suitable for determining a radiation dose emitted by the first material 115. In such embodiments, the measuring device 117 may be located on or integrated into the container 110 such that as the container device is received by the mixing device 130 of the system, the measuring device may be in communication with a measuring port disposed on an exterior surface of the mixing device 130. In other embodiments of the present invention, the container 110 may alternatively comprise a measuring port such that as the container device is received by the mixing device 130 of the system, the measuring port may be in communication with a measuring device 134 disposed on an exterior surface of the mixing device 130. The measuring device 117,134 utilized in the various embodiments of the present invention may comprise various types of radiation detection sensors, including, but not limited to, a dosimeter, Geiger counter, and/or other radiation detection means. One skilled in the art will appreciate that the measuring device 117, 134 may comprise different types of radiation detection sensors suitable for measuring the radiation dose produced by various types of, for instance, radiopharmaceutical agents, that may be utilized in the embodiments of the present invention. In addition, according to some other embodiments of the present invention, the measuring device 117, 134 may comprise a radiation detection sensor operably engaged with a digital circuit so as to provide digital information to the computer device 135 corresponding to the radioactivity level of the first material such that the predetermined ratio may be selected to produce a mixture having a predetermined radioactivity level. As described below, the computer device 135 may be operably engaged with and in communication with the dispensing device 120, according to some embodiments of the present invention.

The container 110 may also further comprise a first memory device 118 operably engaged therewith. The first memory device 118 may be configured to receive a data set related to the first material, wherein the data within the data set related to the first material may include, but is not limited to: radiation dose of the first material at its time of fabrication, method of fabrication (such as cyclotron or irradiating device), time of fabrication, type of substance (such as, for example, FDG), and other data related to the first material. In one alternative embodiment, the data set related to the first material may be transferred to the first memory device 118 by a wire-based electronic connection such as USB port, or other physical wire connection. In other embodiments, the data set related to the first material may be transferred to the first memory device 118 by wireless methods including, but not limited to, radio frequency (RF), infra-red (IR), bluetooth or other wireless methods. The data set related to the first material stored in the first memory device 118 may be useful, for instance, in aiding the operator of the system in identifying the first material and the characteristics thereof, by, for instance, interrogating the first memory device 118, using for instance RFID technology, wire-based electronic connection, or other suitable connection to an electronic device adapted to display the data set related to the first material upon electronically interrogating the first memory device 118. In one alternative embodiment of the present invention, the computer device 135 may be in communication with the mixing device 130 of the present invention and may be configured to be capable of interrogating the first memory device 118 to access the data set related to the first material contained within the container device 110 so as to be capable of cross-checking, for example, the expected radiation dose of the first material (which may be calculated by the computer device 135, for example, based on the known half-life of the first material and the radioactivity level of the first material at the time it was placed in the container device 110). Thus, the computer device 135 may be adapted for comparing the calculated expected radiation dose of the first material with the detected radiation level of the first material that may be transmitted by the measuring device 117, 134 that may be operably engaged with the container device 110 in order to determine the substantially real-time radiation level of the first material.

According to some embodiments of the present invention, a plurality of container devices 110 may be used to contain a corresponding plurality of first materials which may comprise a variety of different materials, components, mixtures, or other compounds suitable for administering to an individual as part of a diagnostic, therapeutic, and/or medical procedure. In addition, the mixing device 130 described herein may be capable of operably engaging the plurality of container devices so as to be capable of mixing a plurality of first materials to generate a mixture containing, for example, one to ten (or more) first materials to form a mixture as described herein.

FIG. 1 also shows mixing 130 according to one embodiment of the present invention. The mixing device 130 may operably engage the container device 110 as shown in FIG. 1 such that the valve 114 and aperture 112 of the container device 110 become operably engaged with a fluid port 132 defined in an exterior surface of the mixing device 130. The fluid port 132 may further operably engage the valve 114 such that the first material 115 may pass through the valve 114 and aperture 112 of the container device 110 and into the mixing device 130 as shown in FIG. 1. The fluid port 132 may further comprise, for instance, a circuit or electromechanical valve device (such as a solenoid valve, for example) configured to actuate the valve 114 of the container device 110 as it is received by the mixing device 130.

The mixing device 130 may be configured to mix at least a portion of the first material 115 with at least a portion of the second material 145. The first material may comprise, for instance, a radiopharmaceutical agent that is fabricated in a cyclotron or irradiating device and received from the container device 110 previously described. The second material 145 may comprise a diluent, such as, for instance, intravenous saline solution that may be suitable for diluting the first material. The second material 145 may be provided by the mixing device 130 via a second container 140 configured to hold the second material and in some embodiments, operably engaged with the mixing device. In other embodiments, the second material 145 may be provided by the mixing device 130 via a separate reservoir or other fluid system in fluid communication with the mixing device 130. The mixing device 130 may further comprise an internal tubing set 133 configured to mix at least a portion of the first material 115 with at least a portion of the second material 145 according to a predetermined ratio, to form a mixture 125. In one embodiment, the internal tubing set 133 may further comprise disposable polymer tubing to be replaced between each mixing cycle or at selected intervals such that, in some embodiments, a newly provided disposable sterile internal tubing set 133 may be operably engaged with the mixing device 130 prior to each subsequent mixing operation. The internal tubing set 133 may further be in communication with the fluid port 132, container device 110, second container device 140 and via a plurality of valve mechanisms actuated via electromechanical devices that may be controlled by the computer device 135 described generally below.

The mixing device 130, according to some embodiments of the present invention, may further comprise a computer device 135 that may be operably engaged with the mixing device 130 and capable of cooperating with the mixing device 130 and/or measuring device 117,134 to form a mixture 125. The mixture 125 may be formed by mixing at least a portion of the first material 115 with at least a portion of a second material 145 that may be provided by the mixing device 130, according to a predetermined ratio to form the mixture 125. According to some embodiments of the present invention, the computer device 135, in cooperation with the measuring device 117, 134 may determine the radiation dose emitted by, for instance, a radiopharmaceutical agent or other component of the first material 115, and in response, adjust the predetermined ratio to provide a mixture having a selected radiation dose amount. In other embodiments, the computer device 135 may be further capable of receiving an input from an operator, wherein the input may comprise, for instance, the selected radiation dose amount. The computer device may further be capable of receiving an input comprising, for instance, an individual data set that is related to an individual to whom the mixture 125 (such as, for instance, a diluted radiopharmaceutical agent) is to be dispensed during a medical procedure such as, for example, a PET imaging procedure and/or other imaging modalities. The individual data set may comprise, for instance, weight of the individual, height of the individual, time and date of the procedure, patient identification number, and other information that may be used to calculate, for instance, an appropriate radiation dose amount. One skilled in the art will appreciate that the selected radiation dose amount of the mixture 125 may be attained by mixing a first material 115 comprising for instance, a radiopharmaceutical agent characterized by a radiation dose, with a second material, comprising for instance, an intravenous saline solution, such that the first and second materials are mixed according to a predetermined ratio. One skilled in the art will also appreciate that the selected radiation dose amount of the mixture 125 may vary depending on various factors related to the type of imaging, diagnostic, and/or therapeutic procedure being performed, the size of the individual to which the mixture 125 is being dispensed, and other factors, including, but not limited to, the radiation dose of the first material 115 prior to forming the mixture 125.

The computer device 135 may be further adapted to communicate with a data port 136 that may be disposed, for instance, on an outer surface of the mixing device 130 so as to communicate with a second memory device 127 that may be operably engaged with a dispensing device 120. The dispensing device 120 may hold the mixture 125 formed by the mixing device 130 of the present invention as described more specifically below. The data port 136 may further comprise, for instance, a physical electronic connection between the mixing device 130 and the second memory device 127. According to other embodiments, the data port 136 may comprise a transceiver for sending data to the second memory device 127 via wireless methods such, as for instance, radio frequency (RF) techniques, infra-red (IR) connections, bluetooth and/or other suitable wireless methods.

FIG. 1 also shows a dispensing device 120 according to one embodiment of the present invention. The dispensing device 120 may be configured to hold the mixture 125 formed by the mixing device 130 of the system. Furthermore, in other embodiments of the present invention, the dispensing device 120 may be in communication with the mixing device 130 as shown in FIG. 1, for example, so as to reduce the handling of the mixture 125 contained within the dispensing device 120. In other embodiments, the dispensing device 120 may be in fluid-tight fluid communication with the tubing set 133 and mixing device 130 such that the mixture 125 formed by the mixing device 130 may be directed to the dispensing device (which may further comprise shielding 123) for dispensation thereof. Minimal handling of and exposure to the mixture 125 may be preferable in some cases, since the mixture 125, as described above, may comprise a combination of at least a portion of the first material 115, which may comprise, for instance a radiopharmaceutical agent, and a second material 145, such as, for instance an intravenous saline solution. As such, the mixture 125 may be adapted to produce a selected radiation dose amount that, if unshielded, may be harmful to an operator of the system. In addition, and as described herein, the first material 115 may also contain one or more pharmaceutical agents having toxic or other adverse chemical properties such that the components of the present invention may be useful for minimizing handling of the materials and/or mixtures utilized in the various embodiments of the present invention.

As described above, the dispensing device 120 may further comprise a second memory device 127 configured to receive a data set related to the mixture 125, wherein the data within the data set related to the mixture may include: selected radiation dose amount of the mixture at its time of formation in the mixing device 130, classification of first material 115 used to form the mixture 125 (such as, for example, FDG), and other data related to the mixture 125. The data set related to the mixture may be transferred to the second memory device 127 from the data port 136 by a wire-based electronic connection such as USB port, or other physical wire connection. In some embodiments, the data set related to the mixture may be transferred to the second memory device 127 from the data port 136 by wireless methods including, but not limited to, radio frequency (RF), infra-red (IR), bluetooth or other suitable wireless methods. The data set related to the mixture 125 stored in the second memory device 127 operably engaged with the dispensing device 120 may be useful, for example, in aiding the operator of the system in identifying the mixture and the characteristics thereof, by, for example, interrogating the second memory device 127, using technologies including, but not limited to, RFID technology, wire-based electronic connection, or any other suitable connection to an electronic device adapted to display the data set related to the mixture upon electronically interrogating the second memory device 127.

The dispensing device 120, as shown in FIG. 1, may also comprise shielding 123 which may include, but is not limited to, a lead, tungsten, or polycarbonate layer (or any combination thereof) having a thickness suitable for shielding an operator of the system from the predetermined radiation dose amount produced by the mixture 125 contained within the dispensing device 120. One skilled in the art will appreciate that the shielding 123 may vary in material, thickness, and other parameters in order to provide reduce an individual's exposure to a variety of radiation, including, but not limited to, x-rays, beta radiation, gamma radiation, and other radiation types having varying strengths, depending on the characteristics of the mixture 125 contained within the dispensing device 110.

According to another embodiment, the dispensing device 120 may further comprise a cartridge device 121 for holding the mixture 125. In this embodiment, the cartridge device 121 may be placed within the shielding 123 of the dispensing device 120 so as to provide shielding from radiation emissions that may be produced by the mixture 125 which may further comprise diluted pharmaceutical and/or radiopharmaceutical agents of various types. The cartridge device 121 may further comprise, for example, a vial or syringe composed of polymer, glass, metal, or any other material suitable for containing the mixture 125. Furthermore, the cartridge device 121 may be, in some embodiments, reusable for multiple injection iterations in the system of the present invention. According to other embodiments, the cartridge device 121 may be disposable, and thus discarded and replaced after the mixture 125 is dispensed from the dispensing device 120 as described herein.

The dispensing device 120 may, in some embodiments, be an injection device adapted to inject at least a portion of the mixture 125 into an individual, such as, for example, an individual undergoing a PET imaging procedure. The dispensing device 120 further comprising the shielding 123 operably engaged therewith may be used, for example, with a hand-operated injection unit, adapted to inject at least a portion of the mixture 125 into an individual. The shielding 123 may protect, for example, an operator of the system, from a radiation dose emitted from the mixture 125 as at least a portion of the mixture is hand-injected into the individual. In embodiments of the present invention wherein the dispensing device 120 can also function as an injection device, one skilled in the art will appreciate that the dispensing device may further comprise, for instance, a syringe mechanism, compatible with a hand-injector, and/or power injector device 210 as described herein. In other embodiments, the dispensing device may further be suitable for dispensing the mixture to an individual for oral ingestion.

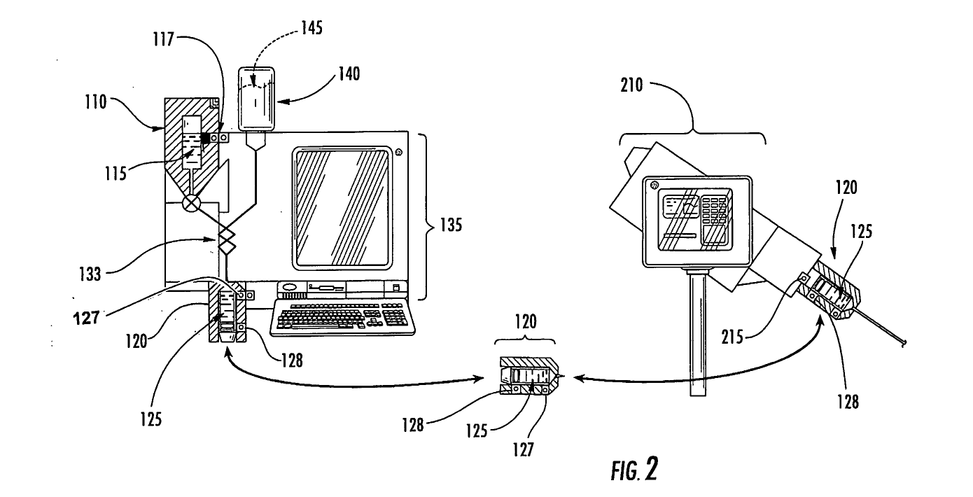

As shown in FIG. 2, according to other embodiments, the dispensing device 120 may be used with a power injector device 210. In such embodiments, the power injector device 210 may receive the dispensing device 120 as shown in FIG. 2, wherein the power injector device 210 may actuate the dispensing device 120 to inject the mixture 125 into an individual while the operator of the system is positioned in a remote location, such as, for instance, a control room adjacent to an imaging room wherein the individual may be positioned during the course of an imaging procedure.

Furthermore, the power injector device 210 may interrogate and/or receive a data set related to the mixture 125 from, for example, the second memory device 127 that may be operably engaged with the dispensing device 120, as described herein. In such embodiments, the power injector device 210 may identify the mixture 125 held by the dispensing device 120 so as to ensure that the selected radiation dose amount is provided to the individual, in light of the procedure type and the data related to the mixture 125.

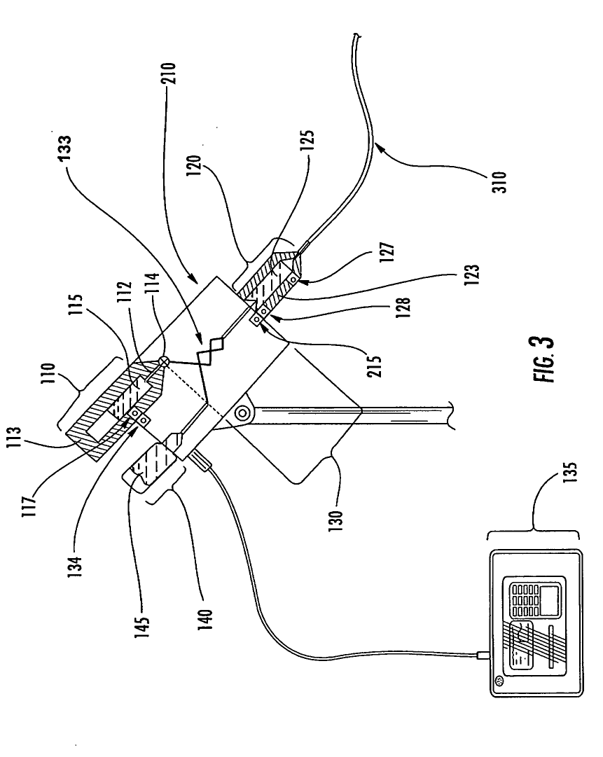

For embodiments of the present invention where the dispensing device 120 is compatible with, for instance, a power injector 210, the dispensing device, may further comprise a measuring port 128, compatible, for instance, with a measuring device 215 that may be operably engaged with the power injector device 210 as shown in FIG. 2. According to some embodiments (such as the integrated mixing device 130/computer device 135/automated injector device 210 embodiment shown generally in FIG. 3), the measuring port 128 of the dispensing device 120 may comprise a second radiation measurement device (including, but not limited to a dosimeter or digital radiation measurement sensor) for determining a radioactivity level of the mixture 125 and for transferring the determined radioactivity level of the mixture 125 to the computer device 135 such that the automated injector device 210 may be capable of dispensing a dose of the mixture to the individual corresponding to a predetermined radiation dose.

According to other embodiments of the present system, a power injector device 210 may be operably engaged with the dispensing device 120 (such as a syringe functionally encapsulated as part of the dispensing device 120). Furthermore, the power injector device may include its own radiation measuring device 215 that may measure the radiation dose of a mixture 125 comprising, for instance, one or more pharmaceutical agents and/or radiopharmaceutical agents. The pharmaceutical agents within the mixture 125 may be held within the dispensing device 120 such that the power injector device 210 can inject at least a portion of the mixture 125 into an individual in the form of, for instance, a predetermined radiation dose amount. One skilled in the art will appreciate that the power injector device 210 may further comprise, for example, a computer device, display, control systems, and other components necessary to automatically purge a connection with the individual prior to injecting the mixture 125, and automatically inject the mixture 125 according to a pre-defined flow rate and volume such that at least a portion of the mixture 125 is automatically injected into the individual.

According to some embodiments of the present invention, a plurality of dispensing devices 120 may be operably engaged with the mixing device 130 so as to be capable of receiving a corresponding plurality of mixtures that may be formed by the present invention. Thus, the mixing device 130 described herein may be capable of operably engaging the plurality of dispensing devices so as to be capable of dispensing the corresponding plurality of mixtures to one or more individuals (such a single mixing device may be capable of centrally administering mixtures to a plurality of different individuals). In other embodiments, the plurality of dispensing devices may be utilized to dispense the corresponding plurality of mixtures to a single individual as part of a therapeutic, diagnostic, and/or medical procedure requiring the dispensation of a plurality of mixtures containing, for example, one or more first materials. For example, the mixing device 130 may be operably engaged with one to ten (or more) dispensing devices 120 so as to be further capable of providing a corresponding plurality of mixtures to one or more individuals in a diagnostic, therapeutic, and/or medical procedure.

In other embodiments of the invention, such as those shown generally in FIG. 3, the mixing device 130 of the present invention may be integrated with the power injector device 210. Furthermore, as described herein, the mixing device 130 may comprise a computer device 135 for controlling both the mixing device 130 and the power injector device 210 so as to inject at least a portion of the mixture 125 formed by the mixing device 130 into an individual.

As described herein, the system of the present invention may comprise a first container device 110 for holding a first material 115. The container device 110 may thus be operably engaged with the integrated power injector device 210 and mixing device 130, described in detail above, so as to reduce manual handling of the first material 115 and to mix at least a portion of the first material 115 with at least a portion of a second material 145 according to a predetermined ratio to form the mixture 125. Furthermore, because the mixing device 130 and power injector device 210 are integrated in such embodiments, handling of the mixture 125 may also be reduced prior to injection of the mixture 125 into an individual. According to some embodiments of the present invention, the automated injector device 210 (and the integrated mixing device 130 included therein) may further comprise a disposable sterile tubing set 133 (or other internal tubing set 133) for mixing at least a portion of the first material 115 (such as a radiopharmaceutical agent or other pharmaceutical agent) with at least a portion of the second material 145 (such as an intravenous saline solution) to form a mixture 125 having a predetermined ratio of first material to second material. The system of these embodiments may further comprise multiple container devices 110/140 configured to hold various types of first materials 115 and second materials 145. The container devices 110, 140 may be engageable with the automated injector device for providing the various materials thereto with minimal handling. Furthermore, the power injector device 210 may be capable of injecting the mixture 125 into an individual via a dispensing device 120 selectively operably engaged with the power injector device 210.

In addition, the dispensing device 120 (including, but not limited to a syringe) may be operably engaged with the power injector device 210 in a substantially fluid-tight manner so as to reduce manual handling of the mixture contained within the dispensing device 120. For example, the dispensing device 120 may be fitted with a fluid-tight threaded connection or luer fitting for connecting (via a fluid-tight seal) to the dispensing device 210. Furthermore, the dispensing device 120, as described herein with respect to other system embodiments of the present invention, may also comprise a second shielding device 123 operably engaged therewith for shielding a user from emissions originating from the mixture 125 contained within the dispensing device 120. According to some embodiments, the dispensing device 120 may be capable of operably engaging (via a luer lock or other fluid-tight connection known to those skilled in the art) an intravenous line 310 capable of carrying the mixture 125 from the dispensing device 120 to an individual (such as a human patient awaiting an imaging procedure that is dependent upon the injection of the mixture 125 in order to produce a medical image of the individual).

Furthermore, as shown in FIG. 3, the system of the present invention may further comprise a computer device 135 operably engaged with the automated injector device 210 and configured to cooperate with the automated injector device 210 to form the mixture 125 according to the predetermined ratio (that may be computed by the computer device 135 in response to a received dosage information input by a clinician, for example). Furthermore, the first container device 110 (containing the first material 115 (such as a radiopharmaceutical)), may further comprise a first radiation measurement device 117 operably engaged therewith for determining a radioactivity level of the first material 115 and for transferring the determined radioactivity level of the first material 115 to the computer device 135. For example, the first radiation measurement device 117 may comprise a digital radiation sensor that is capable of measuring the radioactivity level of the first material and thereafter transferring the radioactivity level (via a data port 134 included in the automated injector device 210) to the computer device 135 that may be operably engaged with the automated injector device 210. Thus, the computer device 135 may be capable (using the determined radioactivity level of the first material) of producing a mixture 125 having a selected radioactivity level by selecting an appropriate predetermined ratio to produce a mixture 125 having a selected resulting radioactivity level.

According to some embodiments of the present invention, the dispensing device 120 comprises a second radiation measurement device (integrated with, for example, the measurement port 128) operably engaged therewith for determining a radioactivity level of the mixture 125 and for transferring the determined radioactivity level of the mixture to the computer device 135 such that the power injector device 210 may be capable of dispensing a dose of the mixture 125 to the individual corresponding to a predetermined radiation dose. In one alternative embodiment, the computer device 135 may, in response to the determined radioactivity level of the mixture 125, direct the automated injector device 210 to adjust the overall amount of the mixture 125 that is administered to the individual via the dispensing device 120. Furthermore, the computer device 135 may also direct the automated injector device 210 to adjust the predetermined ratio of the mixture 125 by increasing the amount of first material 115 and/or second material 145 that is present in the resultant mixture 125 until a selected radiation dose is achieved and confirmed by the second radiation measurement device 128.

Additionally, such a power injector device 210 may further comprise, for instance, multiple dispensing cartridges or syringes containing, mixtures, flushing agents (including, but not limited to, intravenous saline solution, water, or other suitable diluents), first materials 115 and/or second materials 145. The outlet of each cartridge may be in fluid communication to the line 310 delivering the pharmaceutical agent, radiopharmaceutical agent, or other mixture 125 to the patient for diagnostic, therapeutic, imaging, dilution, and/or flushing purposes. Such a line would assure that any volume of material in the tube going to the patient is effectively utilized.

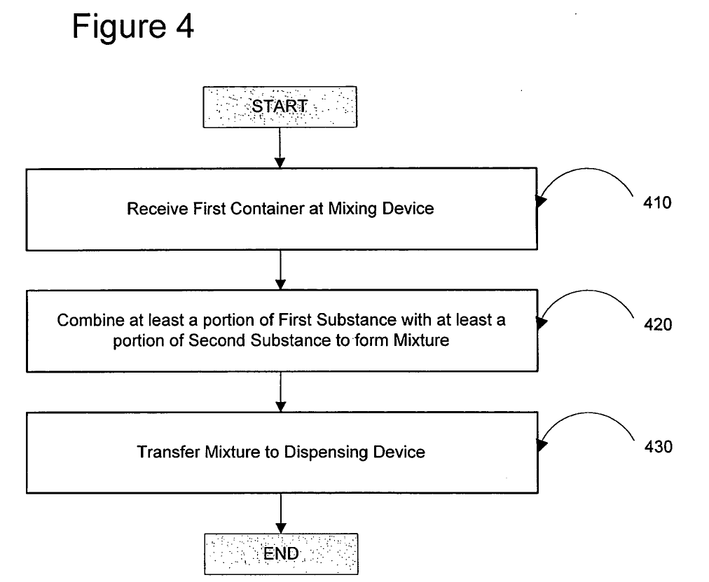

FIGS. 4-8 illustrate several non-limiting exemplary method embodiments for forming a mixture 125 by mixing, for instance, a first material 115 with a second material 145 using a mixing device 130 such as that disclosed generally above with regard to the system embodiments of the present invention. The resulting mixture 125 may also comprise other ingredients as well. First, as shown in FIG. 4, in step 410, the container device 110, holding the first material 115, is received by the mixing device 130. The receiving step 410 may further comprise, for instance, measuring the radiation dose of a first material 115 held in the container device 110 using, for instance, a measuring device 117, 134 (including, but not limited to a dosimeter or digital radiation sensor capable of communicating with a computer device 135 that may be operably engaged and/or in communication with the mixing device 130).

In step 420, the mixing device 130 mixes at least a portion of the first material 115 with at least a portion of the second material 145 according to a predetermined ratio to form the mixture 125. The mixing step 420 may further comprise, for instance, the steps of receiving the dispensing device 120 configured to hold the mixture 125; determining the predetermined ratio using a selected radiation dose amount input by an operator of the system; and determining the radiation dose of the first material 115 measured by, for instance, the measuring device 117, 134 in step 410. The mixing step 420 includes, for instance, mixing at least a portion of the first material 115 with at least a portion of the second material 145 according to a predetermined ratio in the internal tubing set 133 of the mixing device 130.

Step 430 comprises transferring the mixture 125 to the dispensing device 120. Step 430 may occur automatically as the mixing device 130 forms the mixture 125 and transfers the mixture to the dispensing device 120 received in the mixing device 130 as shown in FIG. 1. For example, step 430 may comprise filling a shielded syringe or other dispensing device 120 with a mixture 125 formed by the mixing device 130 or automated injector device 210 of the present invention.

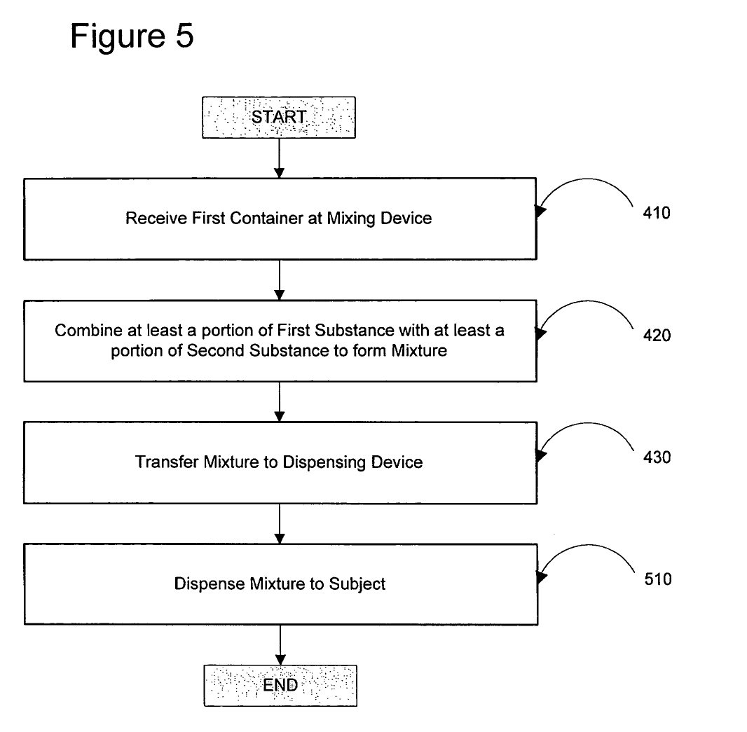

As shown in FIG. 5, the method of the present invention may, in some embodiments, further comprise dispensing the mixture 125 to an individual (including, but not limited to a patient awaiting a subsequent medical imaging procedure) as shown generally in step 510. Step 510 may comprise actuating an automated injector device 210 (including, but not limited to the automated injector device 210 shown in FIG. 3) to inject the mixture 125 (contained in a syringe or other dispensing device 120) into an individual. Step 510 may, in some embodiments, comprise injecting the mixture 125 directly from the dispensing device 125 via a needle and/or indirectly via an intravenous line 310 as shown in FIG. 3 or other suitable means). In some embodiments, a separate mixing device 130 and automated injector device 210 (including, but not limited to those shown generally in FIG. 2) may be utilized. The method may further comprise disengaging the dispensing device 120 from the mixing device 130 (subsequent to the production of the mixture) and subsequently operably engaging the dispensing device 120 with the separate automated injector device 210 for automatically injecting the mixture into an individual (step 510, for example). The dispensing step 510 may further comprise engaging the dispensing device 120 described above with a hand-injector and/or syringe device for manual injection into an individual.

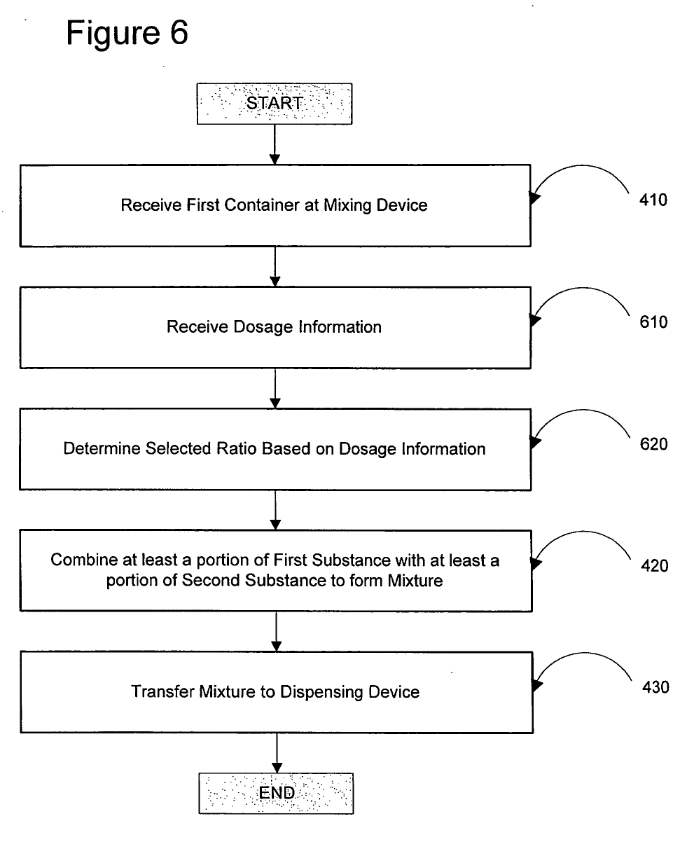

Referring to FIG. 6, the method embodiments of the present invention may further comprise several dosage calculation steps prior to controlling the mixing device 130 (or automated injector device 210, as shown in FIG. 3) to mix the first material 115 and second material 145 to form the mixture 125. For example, step 610 comprises receiving dosage information corresponding to a selected dose of the mixture 125 to be dispensed to an individual. For example, step 610 may comprise receiving (via the computer device 135 or other user interface in communication with the mixing device 130 and/or automated injector device 210) dosage information (including, but not limited to a total desired radiation dose in millicuries) that may be input by a clinician or health physicist prior to the initiation of the production of the mixture 125 to be administered to the individual. Furthermore, as shown in step 620, the method may further comprise determining the predetermined ratio prior to the controlling step based on the received dosage information. In one embodiment, the computer device 135, mixing device 130, and/or automated injector device 210 may be configured to be capable of determining the predetermined ratio of first material 115 to second material 145 (including, but not limited to, saline, water, or other suitable diluent) to form a mixture 125 that is capable of providing the selected dosage based on the radioactivity, half-life, and decay time information for a given radiopharmaceutical that may be present in the first material 115.

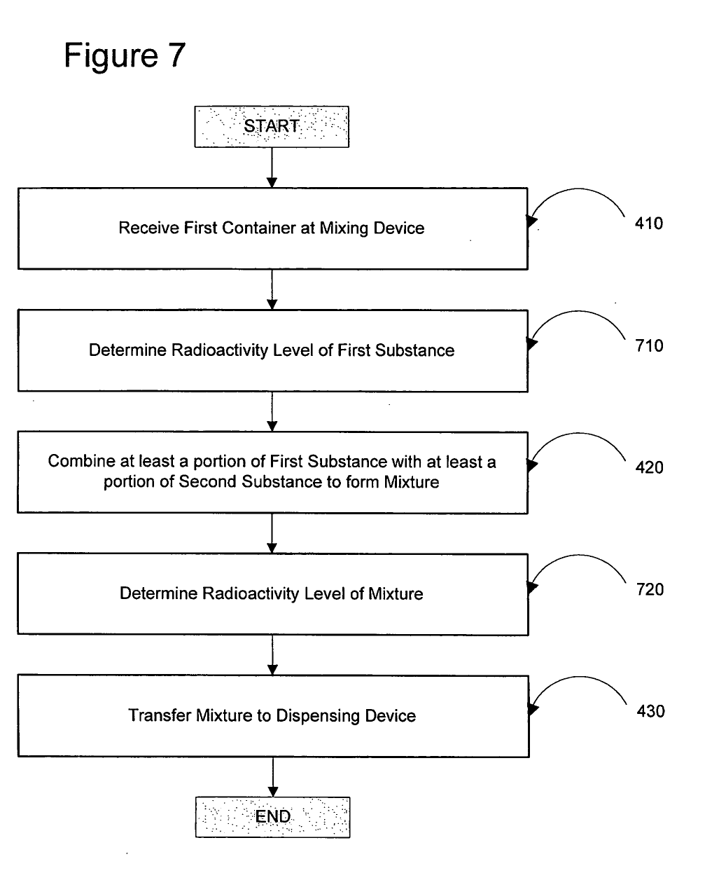

Thus, as shown in FIG. 7, the computer device 135 of the system embodiments of the present invention may interrogate the first radiation measurement device 117 and/or the first memory device 118 included as part of the first container device in order to determine baseline radiation, half-life, and/or identification information related to the first material 115, as shown in step 710. Thus according to method step 710, the computer device 135 may be capable of determining the predetermined ratio based on a selected dosage or dose rate that may be input by an individual and received as part of step 610 as well as the baseline radiation, half-life, and/or identification information related to the first material 115. Furthermore, as shown in FIG. 7, the method of the present invention may also comprise determining a radioactivity level of the mixture 125 prior to the directing step (step 430, for example) such that the mixture 125 may be dispensed to an individual so as to expose the individual to a selected radiation dose that may be input by an individual, including but not limited to a clinician or heath physicist, as part of step 610 (shown in FIG. 6).

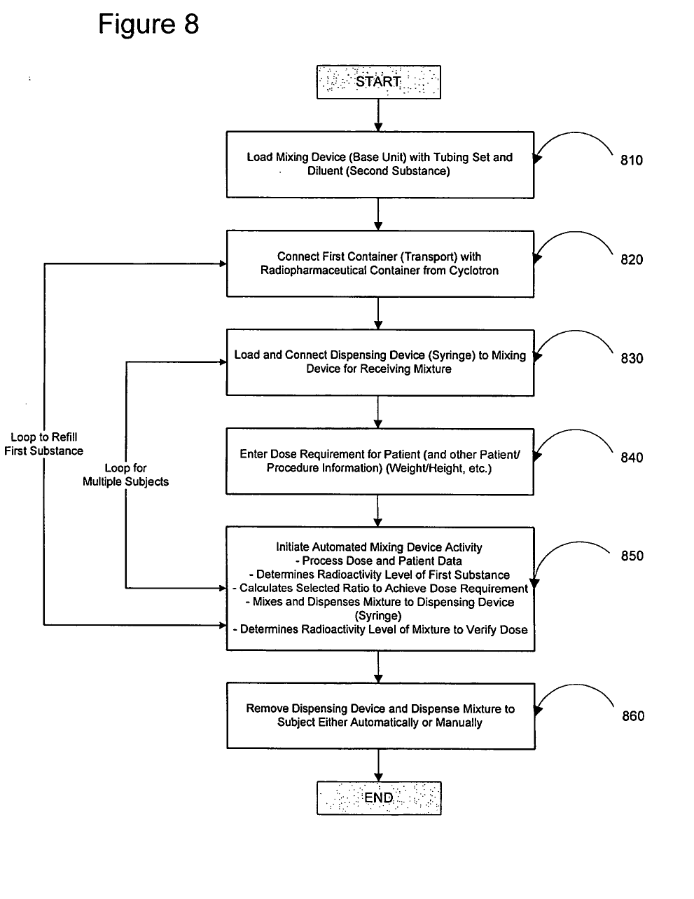

FIG. 8 shows an exemplary embodiment of the present invention suitable for mixing a first material comprising a radiopharmaceutical with a second material comprising a diluent. Step 510 comprises loading the mixing device 130 (denoted as the “base unit”) with diluent (the second material 145) and an internal tubing set 133 (such as a disposable sterile tubing set 133, as described herein). Step 820 comprises connecting the container device 110 with a cyclotron container, containing, for instance, radiopharmaceutical agents. Step 830 comprises loading and connecting the dispensing device 120 (including, but not limited to a fluid-tight syringe) to the mixing device 130. In step 840, data related to the imaging procedure may be entered into the computer device 135 of the present invention. Step 850 which may be performed by the mixing device 130 in cooperation with the computer device 135 operably engaged therewith, may comprise automatically processing the data related to the imaging procedure, measuring a radiation dose emitted by the radiopharmaceutical (contained, for example in the first material 115 and measured by the first radiation measurement device 117 operably engaged with the first container device 110), determining the predetermined ratio to achieve a selected radiation dose amount, automatically mixing the first 115 and second materials 145 to form the mixture 125, and verifying the selected radiation dose amount emitted by the mixture 125. Step 860 comprises removing the dispensing device 120 from the mixing device 130 and dispensing the mixture 125 to an individual as part of a medical procedure, including but not limited to: a PET imaging procedure, a CT imaging procedure, an alternate medical imaging procedure, a therapeutic procedure, a diagnostic procedure, and/or other suitable medical procedures requiring the injection of the mixture 125.

In addition to providing systems and methods, the present invention also provides computer program products for performing the operations described above. The computer program products have a computer readable storage medium having computer readable program code means embodied in the medium. With reference to FIG. 1, the computer readable storage medium may be included as part of the computer device 135 in communication with the mixing device 130 and/or automated injector device 210 (as shown generally in FIG. 3), and may implement the computer readable program code means to perform the above discussed operations.

FIGS. 4-8 are non-limiting block diagrams, flowcharts and control flow illustrations of methods, systems and program products according to embodiments of the invention. It will be understood that each block or step of the block diagrams, flowcharts and control flow illustrations, and combinations of blocks in the block diagrams, flowcharts and control flow illustrations, can be implemented by computer program instructions. These computer program instructions may be loaded onto a computer (including, but not limited to the computer device 135 in communication with the mixing device 130 and/or automated injector device 210 described herein with respect to the embodiments of the present invention) or other programmable apparatus to produce a machine, such that the instructions which execute on the computer or other programmable apparatus form means for implementing the functions specified in the block diagrams, flowcharts or control flow block(s) or step(s). These computer program instructions may also be stored in a computer-readable memory that can direct a computer or other programmable apparatus to function in a particular manner, such that the instructions stored in the computer-readable memory may produce an article of manufacture including instruction means which can implement the function specified in the block diagrams, flowcharts or control flow block(s) or step(s). The computer program instructions may also be loaded onto a computer or other programmable apparatus, among other things, to cause a series of operational steps to be performed on the computer or other programmable apparatus. This may produce a computer implemented process such that the instructions which execute on the computer or other programmable apparatus provide steps for implementing the functions specified in the block diagrams, flowcharts or control flow block(s) or step(s).

Accordingly, blocks or steps of the block diagrams, flowcharts or control flow illustrations support, among other things, combinations of means for performing the specified functions, combinations of steps for performing the specified functions and program instruction means for performing the specified functions. It will also be understood that each block or step of the block diagrams, flowcharts or control flow illustrations, and combinations thereof, can be implemented by special purpose hardware-based computer systems which perform the specified functions or steps, or combinations of special purpose hardware and computer instructions.

Other modifications and other embodiments of the invention set forth herein will come to mind to one skilled in the art to which this invention pertains having the benefit of the teachings presented in the foregoing descriptions and on the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Further, throughout the description, where compositions are described as having, including, or comprising specific components, or where processes systems or methods are described as having, including, or comprising specific steps, it is contemplated that compositions or the present invention may also consist essentially or, or consist of the recited components, and that the processes or methods of the present invention also consist essentially or consist of the recited steps. Further, it should be understood that the order of steps or order of performing certain actions are immaterial so long as the invention remains operable. Moreover, two or more steps or actions may be conducted simultaneously with respect to the invention disclosed herein.

1. A system for dispensing a mixture into an individual, the system comprising:
at least one container, suitable for holding one or more materials;
at least one dispensing device; and
at least one mixing device for engaging the one or more containers so as to reduce handling of a first one or more materials contained therein and to mix at least a portion of the first one or more materials with at least a portion of a second one or more materials according to a predetermined ratio to form a mixture, the at least one mixing device being capable of directing the mixture to the at least one dispensing device for dispensation thereof into the individual.

2. A system according to claim 1, wherein the at least one mixing device comprises a disposable sterile tubing set for mixing at least a portion of the first one or more materials with at least a portion of the second one or more materials.

3. A system according to claim 1, wherein the at least one container comprises a first memory device configured to be capable of receiving and storing a data set containing data related to the first one or more materials and wherein the at least one mixing device is further capable of interrogating the first memory device to access the data set.

4. A system according to claim 1, further comprising a second container device for holding the second one or more materials and to be engageable with the at least one mixing device for providing the second one or more materials thereto.

5. A system according to claim 1, wherein the at least one mixing device is further capable of engaging the at least one dispensing device so as to supply the mixture thereto.

6. A system according to claim 1, wherein the at least one dispensing device comprises an injection device adapted to inject at least a portion of the mixture into the individual.

7. A system according to claim 6, wherein the at least one dispensing device is capable of operably engaging an automated injector device for injecting at least a portion of the mixture into the individual.

8. A system according to claim 1, further comprising a computer device operably engaged with the at least one mixing device and capable of cooperating with the at least one mixing device to form the mixture according to the predetermined ratio.

9. A system according to claim 8, wherein the computer device is further capable of determining the predetermined ratio.

10. A system according to claim 9, wherein the computer device is further capable of receiving dosage information, the computer device being further capable of determining the predetermined ratio from the dosage information received thereby.

11. A system according to claim 8, wherein the at least one container comprises a first radiation measurement device operably engaged therewith for determining a radioactivity level of the first one or more materials or second one or more materials and for transferring the determined radioactivity level of the first one or more materials or second one or more materials to the computer device such that the predetermined ratio may be selected to produce a mixture having a selected radioactivity level.

12. A system according to claim 8, wherein the at least one dispensing device comprises a second radiation measurement device operably engaged therewith for determining a radioactivity level of the mixture and for transferring the determined radioactivity level of the mixture to the computer device such that the at least one dispensing device may be capable of dispensing a dose of the mixture to the individual corresponding to a selected radiation dose.

13. A system according to claim 1, wherein the at least one container further comprises a first shielding device operably engaged therewith for shielding a user from the first one or materials or the second one or more materials contained therein.

14. A system according to claim 1, wherein the at least one dispensing device further comprises a second shielding device operably engaged therewith for shielding a user from the mixture contained therein.

15. A system according to claim 1, wherein the dispensing device comprises a syringe.

16. A system for dispensing a mixture into an individual, the system comprising:
a first container for holding a first material; and
an automated injector device capable of engaging the container so as to reduce manual handling of the first material contained therein and to mix at least a portion of the first material with at least a portion of a second material according to a predetermined ratio to form a mixture, the automated injector device further capable of injecting the mixture into the individual by a dispensing device selectively operably engaged with the automated injector device.

17. A system according to claim 16, wherein the automated injector device comprises a disposable sterile tubing set for mixing at least a portion of the first material with at least a portion of the second material.

18. A system according to claim 16, wherein the first container device comprises a first memory device capable of receiving and storing a data set containing data related to the first material and wherein the automated injector device is further capable of interrogating the first memory device to access the data set.

19. A system according to claim 16, wherein the dispensing device is capable of operably engaging the automated injector device in a substantially fluid-tight manner to reduce manual handling of the mixture contained therein.

20. A system according to claim 16, wherein the dispensing device comprises a syringe.

21. A system according to claim 16, further comprising a third container device for holding the second material and for selectively engaging the automated injector device for providing the second material thereto.

22. A system according to claim 16, further comprising a computer device operably engaged with the automated injector device and configured to cooperate with the automated injector device to form the mixture according to the predetermined ratio.

23. A system according to claim 22, wherein the computer device is further capable of determining the predetermined ratio.

24. A system according to claim 23, wherein the computer device is further capable of receiving dosage information, the computer device being further capable of determining the predetermined ratio from the dosage information received thereby.

25. A system according to claim 22, wherein the first container comprises a first radiation measurement device operably engaged therewith for determining a radioactivity level of the first material or the second material and for transferring the determined radioactivity level of the first material or second material to the computer device such that the predetermined ratio may be selected to produce a mixture having a selected radioactivity level.

26. A system according to claim 22, wherein the dispensing device comprises a second radiation measurement device operably engaged therewith for determining a radioactivity level of the mixture and for transferring the determined radioactivity level of the mixture to the computer device such that the automated injector device may be capable of dispensing a dose of the mixture to the individual corresponding to a selected radiation dose.

27. A system according to claim 16, wherein the first container device further comprises a first shielding device operably engaged therewith for shielding a user from the first material or the second material contained therein.

28. A system according to claim 16, wherein the dispensing device further comprises a second shielding device operably engaged therewith for shielding a user from the mixture contained therein.

29. A method for dispensing one or more mixtures into an individual, the method comprising:
operably engaging at least one container for holding a first one or more materials with at least one mixing device, so as to provide the first one or more materials to the mixing device and reduce handling of the first one or more materials contained in the container device;
mixing, with the mixing device, at least a portion of the first one or more materials and at least a portion of a second one or more materials according to a predetermined ratio to form the one or more mixtures; and
directing the one or more mixtures to a dispensing device, the dispensing device being capable of dispensing the mixture into the individual.

30. A method according to claim 29, further comprising dispensing the mixture by injecting the mixture into the individual with the dispensing device.

31. A method according to claim 29, further comprising operably engaging the dispensing device with an automated injector device for automatically injecting the mixture into the individual.

32. A method according to claim 29, further comprising receiving dosage information corresponding to a selected dose of the mixture to be dispensed to the individual.

33. A method according to claim 32, further comprising determining the predetermined ratio prior to the mixing step based on the received dosage information.

34. A method according to claim 29, further comprising determining a radioactivity level of the first one or more materials or the second one or more materials prior to the mixing step such that the predetermined ratio may be determined to produce a mixture having a predetermined radioactivity level.

35. A method according to claim 29, further comprising determining a radioactivity level of the mixture prior to the directing step such that the mixture may be dispensed to the individual so as to expose the individual to a selected radiation dose.

36. A computer program product capable of controlling a mixing device to form a mixture, the computer program product comprising a computer-readable storage medium having computer-readable program code portions stored therein, the computer-readable program code portions comprising:
a first executable portion for operably engaging a container device for holding a first material with the mixing device so as to supply the first material to the mixing device;
a second executable portion for controlling the mixing device to mix at least a portion of the first material and at least a portion of a second material according to a predetermined ratio to form the mixture; and
a third executable portion for directing the mixture to a dispensing device, the dispensing device being capable of dispensing the mixture into an individual.

37. A computer program product according to claim 36, further comprising a fourth executable portion for dispensing the mixture by injecting the mixture into an individual with the dispensing device via an automated injector device.

38. A computer program product according to claim 36, further comprising a fifth executable portion for receiving dosage information corresponding to a selected dose of the mixture to be dispensed into the individual.

39. A computer program product according to claim 38, further comprising a sixth executable portion for determining the predetermined ratio prior to the second executable portion based on the received dosage information.

40. A computer program product according to claim 36, further comprising a seventh executable portion for determining a radioactivity level of the first material prior to the second executable portion such that the predetermined ratio may be determined to produce a mixture having a selected radioactivity level.

41. A computer program product according to claim 36, further comprising an eighth executable portion for determining a radioactivity level of the mixture prior to the third executable portion such that the mixture may be dispensed to the individual so as to expose the individual to a selected radiation dose.

42. A mixture suitable for dispensing into an individual during a medical procedure, the mixture made by the method comprising:
operably engaging a first container configured to hold a first material with a mixing device, to provide the first material to the mixing device and reduce handling of the first material contained in the container device;
mixing, with the mixing device, at least a portion of the first material and at least a portion of a second material according to a predetermined ratio to form the mixture; and
directing the mixture to a dispensing device, the dispensing device being further capable of dispensing the mixture into the individual.

43. A method for preparing a radiopharmaceutical agent for injection into an individual, the method comprising:
operably engaging at least one container for holding a first material comprising the radiopharmaceutical agent with a mixing device, so as to provide the first material to the mixing device and reduce handling of the first material contained in the container device;
mixing, with the mixing device, at least a portion of the first material and at least a portion of a second material according to a predetermined ratio to form the mixture; and
directing the mixture to a dispensing device, the dispensing device being further configured to be capable of dispensing the mixture into the individual.

44. A system for dispensing a mixture into an individual, the system comprising:
a plurality of containers, suitable for holding a plurality of first materials;
a plurality of dispensing devices; and
at least one mixing device for engaging the plurality containers so as to reduce handling of the plurality of first materials contained therein and to mix at least a portion of the plurality of first materials with at least a portion of a plurality of second materials according to a predetermined ratio to form a plurality of mixtures, the at least one mixing device being capable of directing the plurality of mixtures to the plurality of dispensing devices for dispensation of at least one of the plurality of mixtures into the individual.

45. A system according to claim 44, wherein the at least one mixing device comprises a disposable sterile tubing set for mixing at least a portion of the plurality of first materials with at least a portion of the plurality of second materials.

46. A system according to claim 44, wherein the plurality of containers each comprise a first memory device configured to be capable of receiving and storing a data set containing data related to at least one of the plurality of first materials held in a corresponding at least one of the plurality of containers and wherein the at least one mixing device is further capable of interrogating the first memory device to access the data set.

47. A system according to claim 44, further comprising a plurality of second container devices for holding the plurality of second materials and for engaging the at least one mixing device for providing the plurality of second materials thereto.

48. A system according to claim 44, wherein the at least one mixing device is further capable of engaging the plurality of dispensing devices so as to supply the plurality of mixtures thereto.

49. A system according to claim 44, wherein the plurality of dispensing device comprises a corresponding plurality of injection devices adapted to inject at least a portion of the plurality of mixtures into the individual.
